(19) United States
(12) Patent Application Publication
(10) Pub. No.: US 2023/0190166 A1
(43) Pub. Date: Jun. 22, 2023

(54) DEVICES AND METHODS FOR TISSUE MAPPING

(71) Applicant: CoreMap, Inc., Burlington, MA (US)

(72) Inventor: Peter S. Spector, Colchester, VT (US)

(21) Appl. No.: 18/086,143

(22) Filed: Dec. 21, 2022

Related U.S. Application Data
(60) Provisional application No. 63/292,315, filed on Dec. 21, 2021.

(51) Int. Cl. A61B 5/287; A61L 29/02; A61L 29/14; A61B 5/367; A61B 5/00

(52) U.S. Cl. CPC A61B 5/287; A61L 29/02; A61L 29/14; A61B 5/367; A61B 5/6852; A61B 2562/164; A61L 2400/16; A61B 2562/0209; A61N 1/362

(57) ABSTRACT

The present invention provides a device and method for monitoring bioelectrical signals in cells, tissues, and/or organs. The invention makes use of a unique and innovative retractable mapping catheter and electrode array system that has maximum flexibility to conform to the shape of the tissue substrate upon which it is placed. The flexible nature of the catheter and electrode array system allows the electrodes to adapt to the configuration of the tissue upon which it is placed, thus each electrode in the array is in continuous contact with the substrate.

### FIELD OF INVENTION

The present disclosure relates to methods and systems for tissue mapping.

### BACKGROUND

Atrial fibrillation, which accounts for almost one-third of all admissions to a hospital for a cardiac rhythm disturbance, is an uncontrolled twitching or quivering of muscle fibers (fibrils) resulting in an irregular and often rapid heart arrhythmia associated with increased mortality and risk of stroke and heart failure. See, e.g., Calkins et al., Treatment of Atrial Fibrillation with Anti-arrhythmic Drugs or Radio Frequency Ablation: Two Systematic Literature Reviews and Meta-analyses, 204) Circ. Arrhythmia Electrophysiol. 349-61 (2009). Atrial fibrillation may be paroxysmal or chronic, and the causes of atrial fibrillation episodes are varied and often unclear; however, atrial fibrillation manifests as a loss of electrical coordination in the top chambers of the heart. When fibrillation occurs in the lower chambers of the heart, it is called ventricular fibrillation. During ventricular fibrillation, blood is not pumped from the heart, and sudden cardiac death may result. Existing treatments for cardiac fibrillation include medications and other interventions to try to restore and maintain normal organized electrical activity to the atria. When medications, which are effective only in a certain percentage of patients, fail to maintain normal electrical activity, clinicians may resort to incisions made during open heart surgery or minimally-invasive ablation procedures, whereby lines of non-conducting tissue are created across the cardiac tissue in an attempt to limit the patterns of electrical excitation to include only organized activity and not fibrillation. Id. at 355. If sufficient and well-placed, the non-conducting tissue (e.g., scar tissue) will interfere with and normalize the erratic electrical activity, ideally rendering the atria incapable of supporting cardiac activity.

Catheter ablation targeting isolation of the pulmonary veins has evolved over the past decade and has become the treatment of choice for drug-resistant paroxysmal atrial fibrillation. The use of ablation for treatment of persistent atrial fibrillation has been expanding, with more centers now offering the procedure. Unfortunately, the success of current ablation techniques for treating cardiac fibrillation is less than desirable, for example, curing only about 70% of atrial fibrillation in patients where ablation is attempted. Id. at 354. In contrast, ablation for treating heart arrhythmias other than fibrillation is successful in more than 95% of patients. Spector et al., Meta-Analysis of Ablation of Atrial Flutter and Supraventricular Tachycardia, 104(5) Am. J. Car diol. 671, 674 (2009). One reason for this discrepancy in success rates is due to the complexity of identifying the ever-changing and self-perpetuating electrical activities occurring during cardiac fibrillation. Without the ability to accurately determine the source locations and mechanisms underlying cardiac fibrillation in an individual patient and develop a customized ablation strategy, clinicians must apply generalized strategies developed on the basis of cardiac fibrillation pathophysiologic principles identified in basic research and clinical studies from other patients. The goal of ablation is to alter atrial physiology and, particularly, electrophysiology such that the chamber no longer supports fibrillation; it is insufficient to simply terminate a single episode. However, ablation lesions have the potential to cause additional harm to the patient (e.g., complications including steam pops and cardiac perforation, thrombus formation, pulmonary vein stenosis, and atrio-esophageal fistula) and to increase the patient's likelihood of developing abnormal heart rhythms (by introducing new abnormal electrical circuits that lead to further episodes of fibrillation). In addition, existing catheters with their associated electrode configurations that are used to assist in preventing, treating, and at least minimizing if not terminating cardiac fibrillation have several shortcomings, including that the electrodes are too large, the inter-electrode spacing is too great, and the electrode configurations are not suitably orthogonal to the tissue surface.

Further complicating the use of conventional contact-based catheters is the fact that some arrhythmias are transient or nonperiodic in nature; therefore, these contact-based catheters are less suitable for mapping these arrhythmias since the sequential contact-based methodology is predicated on the assumption that recorded signals are periodic in nature.

Thus, cardiac fibrillation patients would benefit from new methods and systems for the preventing, treating, and at least minimizing if not terminating cardiac fibrillation in the underlying tissue on which abnormal electrical circuits of reentry are formed which is responsible for the initiation and perpetuation of cardiac fibrillation. These methods and systems would help clinicians minimize or prevent further episodes and increase the success rate of ablation treatments in cardiac fibrillation patients. There remains a need for patient-specific, map-guided ablation strategies that would minimize the total amount of ablation required to achieve the desired clinical benefit by identifying ablation targets and optimizing the most efficient means of eliminating the targets.

### SUMMARY

The present invention provides devices and methods for accurately monitoring electrical signals in cells, tissues, and/or organs for the purposes of mapping, also known as tissue property mapping. The invention makes use of a retractable mapping catheter device with a shape and structure that has sufficient flexibility to conform to the tissue surface upon which it is placed. In cardiac mapping, for example, a physician uses a device of the invention to find the drivers of cardiac arrhythmia in order to treat atrial fibrillation (AF) via more accurate cardiac mapping and ablation. The flexible nature of mapping catheters presented herein allows the catheters to adapt to the configuration of the tissue upon which they are placed.

In a preferred embodiment, a catheter of the invention comprises an electrode array that is placed and held against the cardiac tissue, allowing the physician to construct a high resolution map of the cardiac tissue. The contact of the electrode arrays against tissue allows for a consistent, stabilized signal that reduces noise and allows for more accurate mapping of bioelectrical activity. More accurate mapping means the ability to fully delineate the non-transient electrophysiological properties of the tissue in order to determine where to deliver adequate lesion to treat cardiac fibrillation.

Improving the understanding of tissue's properties enables a more targeted and less extensive ablation strategy, thus improving outcomes for patients with AF undergoing catheter ablation. For these patients, more accurate cardiac tissue mapping means AF drivers are treated with a minimum of ablation lesions, protecting healthy heart tissue from ablation. This is made possible, in part, by the flexible nature of the catheter in addition to using smaller electrodes with closer spacing, automated software allowing rapid data collection and accurate time annotation of electrograms with multiple components.

In one aspect, the invention provides cardiac mapping catheters comprising an electrode array. The electrode array comprises a flexible support frame and a deformable surface, such that, upon deployment from a sheath, the electrode array conforms to the shape of a tissue, allowing the electrode array to lie substantially flat when deployed against the tissue.

In some embodiments, the deformable surface is a flexible membrane. For example, the flexible membrane may include a first layer having a first durometer number, and a second layer having a second durometer number lower than the first durometer number. The second layer of the deformable surface may form a web around the first material.

In some embodiments, the flexible support frame is made of a shape memory metal. Additionally, in some embodiments, the flexible support frame forms a contiguous support for the individual electrodes of the electrode array.

A catheter of the invention may include a plurality of tines. For example, the tines may be flatly shaped such that each tine has a first side and a second side opposite the first side. In this way, the electrodes of the electrode array are positioned apart on the tines of the catheter. The individual electrodes of the electrode array may be positioned on either or both sides of the tines. The electrodes may be positioned as a plurality of single electrodes. Alternatively, the electrodes are positioned as a plurality of electrode pairs arranged in an orthogonal close unipolar configuration. In some embodiments, the electrodes comprising the electrode array include a gold-plated copper trace coated with iridium oxide and are surrounded by an insulating layer.

As previously noted, the invention provides a retractable cardiac mapping catheter device with a shape and structure that has sufficient flexibility to conform to the tissue surface upon which it is placed. The deformable surface of the cardiac mapping catheter is furled when retracted back into a hollow sheath. In some embodiments, the deformable surface is substantially s-shaped when furled within a sheath.

Cardiac mapping catheters of the invention optionally may include two or more magnetic tracking sensors. The magnetic tracking sensors may be positioned on opposing sides of the deformable surface. Using these tracking, or position sensors, the tissue surface is mapped by interpolating one or more positions between the tracking coils. Alternatively, the position of the electrode array is calculated from one or more positions between the tracking coils.

In some embodiments, the electrode array includes a plurality of openings configured to allow for fluid to pass through the array. For example, in some embodiments, the openings are of a diameter sufficient to allow the passage of blood.

The electrode array may also comprise a plurality of pacing electrodes. The pacing electrodes are used to stimulate tissue and produce waves of excitation. In one instance of the invention, the pacing electrodes are used to measure cardiac refractory period.

In other aspects, the invention provides methods for mapping tissue properties. Exemplary methods include providing an electrode array comprising a flexible support frame such that, upon deployment from a sheath, the electrode array conforms to the shape of a tissue and lies substantially flat when deployed against the tissue. When applied to cardiac tissue, the electrodes map electrical activity, for example, during cardiac fibrillation. The electrodes also measure cycle length and identify a minimum cycle length between excitations. The array can create a 3D map of minimum cycle length distribution in the tissue.

In some embodiments of the method, the array identifies a minimum cycle length between excitations via determination of the minimum pacing interval, resulting in local excitation.

In preferred embodiments, the electrode array comprises a plurality of single electrodes of minimal thickness. In other embodiments, the electrode array comprises a plurality of electrode pairs arranged in an orthogonal close unipolar configuration.

### BRIEF DESCRIPTION OF DRAWINGS

FIG. 1 illustrates one embodiment of a cardiac mapping catheter of the invention.

FIG. 2 illustrates one embodiment of electrodes positioned on an electrode array of the invention.

FIG. 3 illustrates dimensions of one embodiment of a cardiac mapping catheter of the invention.

FIG. 4 illustrates one embodiment a cardiac mapping catheter of the invention.

FIG. 5 diagrams a method for mapping cardiac tissue property.

### DETAILED DESCRIPTION

The present disclosure describes catheters comprising a retractable electrode array that improves the mapping of organ tissue electrical impulses in order to more effectively measure, identify, treat, and repair faulty electrical propagation in those organs or tissues. In one embodiment, this disclosure describes novel cardiac mapping catheters that improve the mapping of persistent AF for the purposes of guidance for ablation therapy.

Persistent AF is known to be correlated with shortened cycle length in the atria. By accurately mapping cycle length and conduction velocity and characterizing regional gradients in the atria, optimal target sites for ablation are identified to terminate persistent atrial fibrillation, while sparing tissue from excessive ablation. Treatment of cardiac arrhythmias by catheter ablation includes identifying an area of cardiac tissue having aberrant electrically conductive pathways and then using applied energy to create lesions or scars to isolate or modify the tissue believed to be the source of the arrhythmia. This procedure blocks the abnormal electrical signals. For catheter ablation to be successful, the tissue where the faulty electrical activity occurs must be accurately mapped.

Physicians commonly perform cardiac mapping with catheters that are introduced percutaneously in the heart chambers and which sequentially record the endocardial electrograms. Catheters are small flexible tubes capable of insertion into a blood vessel, for example in the groin, arm or neck, and then threaded into the heart. Cardiac mapping is the process of identifying the temporal and spatial distributions of myocardial electrical potentials during a particular heart rhythm in order to determine the cause of that rhythm. Substrate abnormalities that cause the arrhythmia may manifest as variations in conduction velocity, minimum cycle length, and/or other measurable properties derived from intracardiac signals. Therefore, analysis of signals may be applied in order to deduce tissue property characteristics and arrhythmia driver sites.

Mapping is performed using electrophysiological signals originating from muscular, cardiac or neurological activity. These signals that can be measured through changes in electrical potential across a cell, tissue, or organ.

The present invention provides devices and methods for more accurately monitoring bioelectrical signals in cells, tissues, and/or organs for the purposes of tissue property mapping. Devices of the present invention achieve higher temporal and spatial resolution (including improved signal-to-noise ratio) which improves the understanding of the dynamic behavior of the electrophysiological properties of the tissue. The result is a more accurate mapping of tissue properties, which may include, but are not limited to, wave length, conduction velocity and refractory period. In the case of AF, this enables a more targeted, less extensive ablation strategy.

The invention makes use of a retractable cardiac mapping catheter and electrode array system that has sufficient flexibility to conform to the shape of the tissue upon which it is placed. The flexible nature of the catheter and electrode array system allows the electrodes to adapt to the configuration of the tissue upon which it is placed, thus each electrode in the array is in substantially continuous contact with the tissue. Because the electrodes are in continuous contact with the tissue, a consistent, stabilized signal allows for more accurate mapping of the bioelectrical activity. More accurate mapping means the ability to fully delineate the tissue's fixed and functional electrophysiology in order to deliver adequate lesion sets to the critical arrhythmogenic sites. Improving the understanding of the dynamic behavior of the bioelectric properties of the tissue enables a more targeted and less extensive ablation strategy. This is made possible by the flexible nature of the catheter in addition to using smaller electrodes, as single electrodes or in orthogonal close unipolar pairs, with closer spacing, automated software allowing rapid data collection and accurate time annotation of electrograms with multiple components.

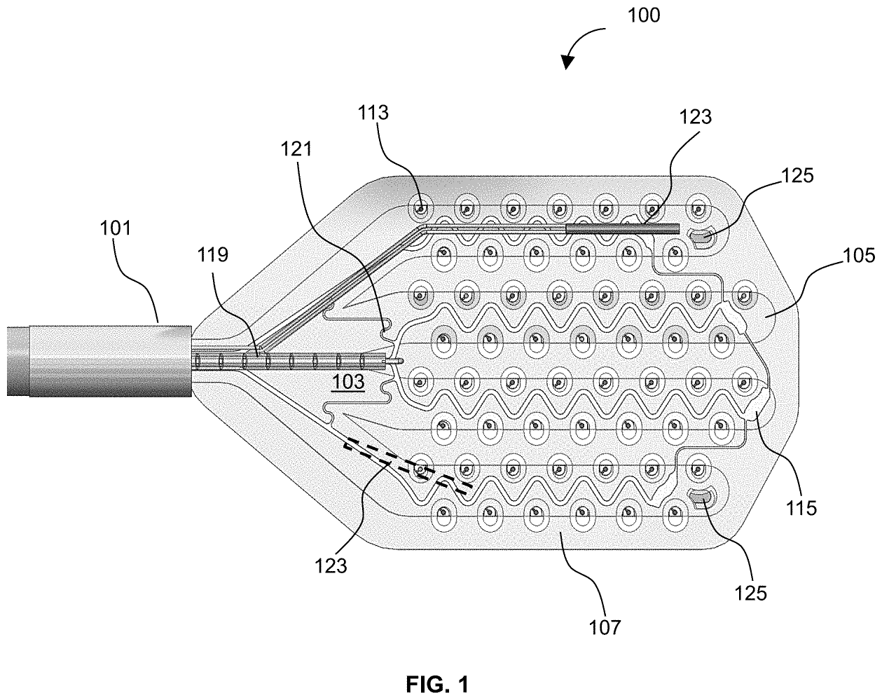

FIG. 1 illustrates one embodiment of a cardiac mapping catheter 100 of the invention. The cardiac mapping catheter 100 may generally include a shaft 101 and an electrode array 103.

Aspects of the invention provide a cardiac mapping catheter that includes an electrode array comprising a flexible support frame 115 and a deformable surface 107, wherein upon retraction from a catheter shaft the electrode array conforms to a shape of a tissue to lie substantially flat when deployed against the tissue.

Cardiac mapping catheters of the invention are capable of being reshaped when it is retracted or withdrawn into a sheath and unfurled when extended from a sheath. When held within a sheath the electrode array 103 may be rolled or furled. When deployed from a sheath, the electrode array 103 unfurls to lie flat against the tissue upon which it is deployed. The flexible and deformable nature of the catheter ensures maximum flexibility to conform to the shape of the tissue upon which it is deployed thus allowing the catheter to lie flat against the tissue. Thus, the deformability and flexibility of the electrode array are enabled by the flexible support frame 115 and the deformable surface 107.

The flexible support frame may be positioned as a contiguous support between individual sensing electrodes 113 of the electrode array. The flexible support frame may be made with composite materials suitable for use in medical devices and characterized by shape memory and super-elastic properties. For example, the flexible support frame may be made of Nitinol, or other alloys of nickel and titanium. The flexible support frame may be designed so that when the electrode array is furled, such as within a sheath, the electrode array is substantially s-shaped, rolled, or folded. After the electrode array is deployed from a sheath, it opens up to an unfurled configuration such that the electrode array lies flat against the tissue upon which it is deployed to match the contours of the tissue. This configuration may be achieved, for example, by using a flexible support frame made of a shape-memory metal.

The deformable surface allows the electrode array to conform to the surface of tissue. The deformable surface may be made from one or more composite materials suitable for use in medical devices and characterized by shape memory and super-elastic properties. These materials may be, for example, Nitinol, other alloys of nickel and titanium, or specialty polymers. In some embodiments, the deformable surface is a flexible membrane.

The deformable surface 107 may include one or more layers of the same or different materials. The deformable surface may be fabricated, for example, as two or more layers, such as by using a first material to develop first layer, then adding to it a second layer using a second material. The deformable surface may have a first layer having a first durometer number, and a second layer having a second durometer number lower than the first durometer number. The use of two materials of different durometers gives the catheter the flexible and bendable nature that allows it to conform to and adhere to the surface of the cardiac tissue thus improving the electrode contact with the tissue surface while also allowing the catheter tip to flatten easily after exiting a catheter sheath. For example, the deformable surface may include a first layer of a suitable non-metallic material such as polyimide, PEEK 450G or nylon with the first layer having a higher durometer number than a second layer material, such as Pebax or Pellethane. In another example, the deformable surface may have a first layer with, for example, PEEK or a thermoplastic polyurethane having a durometer number of 87D, and a second layer of Pebax having a durometer number of 55D. The second layer material may then form a web around the first material. In some embodiments, the second layer may form a web-like structure around the first layer. The web structure may appear as open interstices or may resemble a web, netlike, or netted pattern.

The flexible support frame 115 may be connected to the deformable surface 107 on either side of the deformable surface 107, or it may be retained within the deformable surface. In some embodiments, the cardiac mapping catheter comprises a deformable surface such that the catheter is undulated upon deployment from a sheath and lies flat when deployed against tissue.

Further, the catheter may include a plurality of tines 105 or splines extending from the catheter shaft through the electrode array. For example, the tines may appear as arms or in the form of slender projecting or branching pieces to engage the tissue surface. The tines may be flat on both sides with a rounded edge. Each of the tines may be fabricated within the two-step process and may also have a deformable surface that is able to easily conform to a non-flat surface. In some embodiments, each of the tines or splines may comprise a first side and a second side opposite the first side such that electrodes of the electrode array are positioned apart on the tines. The electrodes may be on the first side, i.e. the top side of the tine or spline and/or on the second side, i.e. the bottom side of the tine or spline.

Each of the two flat sides of the tines may contain a plurality of electrodes such that each electrode 113 may be positioned as groups of adjacent electrodes forming an array. Thus, the tines or splines may comprise a flexible printed circuit (FPC). Further, the tines may include struts. These struts may be in the form of a bar, rod, or other support forming a framework across the tines to resist compression and add stability. In some embodiments, the struts are positioned across the tines or splines.

In some embodiments, a sheath serves as a protective covering for the electrode array until the electrode array is deployed on the tissue surface. When retracted into a sheath, the deformable surface of the electrode array is substantially s-shaped. When deployed from a sheath, the electrode array has a flexible form and is pushed onto the tissue by the deformable nature of the material layers such that the electrode array lies flat against the tissue.

As noted, the materials used in the fabrication of the catheter allow it to adhere to the contours of the cardiac tissue in a way that permits all or a majority of the electrodes to be in constant contact with the tissue. Because of the flexible nature of the catheter, when deployed from a sheath, the tines are able to adhere to the tissue surface. This flexibility allows the clinician to push each electrode against the tissue thus allowing the electrode array to lie flat against the tissue surface. This improves the temporal and spatial resolution, which in turn improves the understanding of the dynamic behavior of the bioelectric properties of the tissue. Beat-to-beat comparison is more accurate because of the stability of the electrodes against the tissue surface. Data from the electrode array can be used to construct a map of cardiac rhythm and tissue properties and can enable a more targeted, less extensive ablation strategy.

The catheter may include a flexible wire fabricated as part of the two-layer process. The flexible wire may extend the length of the second layer of the deformable surface. The flexible wire may be of a type suitable for medical devices. The wire may be controllable and may be used to deform the surface of the catheter to match the surface of the cardiac tissue. By actively modifying the angle of the electrode array (via a pull wire) rather than passively bending it upon contact with the cardiac surface, lateral forces (that would tend to cause the catheter to slide along the surface) are reduced.

In some embodiments, cardiac mapping catheters of the invention include a spine tube 119 in connection with the flexible support frame 115 of the electrode array 103 and a pull wire 121 also in connection with the flexible support frame. In some embodiments, the spine tube 119 is connected to the catheter shaft 101 and the pull wire, and housed within or upon the deformable surface 107. The spine tube 119 may be notched to aid in deflection of the electrode array 103 once the array is deployed from a sheath. In some embodiments, the spine tube is actuated by a mechanism such as a handle or trigger operably connected to the cardiac mapping catheter.

The pull wire may be a deflection pull wire such that when the electrode array is deployed out of a sheath, the deflection wire allows the electrode array to deflect such that the physician can manipulate the electrode array to a desired position upon the tissue. In non-limiting embodiments, the deflection pull wire may deflect the electrode array 90°. A pull wire may be connected to the flexible support frame 115. The pull wire may be actuated by a mechanism in a handle of the cardiac mapping catheter.

In some embodiments, the pull wire is controllable to deform the electrode array to match a cardiac tissue surface. For example, the pull wire is controllable to deploy the electrode array into tortuous anatomical structures to enable mapping of the structures. In some embodiments, the pull wire is controllable to aid in the guidance of the catheter tip into tortuous anatomical structures to enable mapping of the structures. In some embodiments, the combined action of the spine tube and the pull wire are operable to deflect the entire electrode array to a desired position upon tissue such that the electrode array conforms to the contours of the tissue. As noted, in some embodiments, the deformable surface of the electrode array may be furled when the electrode array is retracted back into a sheath. For example, in some embodiments, the deformable surface is substantially s-shaped when furled within a sheath.

Further, the cardiac mapping catheter may include two or more magnetic coils, (e.g., magnetic tracking sensors or magnetic location sensors 123). The magnetic sensors may be positioned on opposing ends of the deformable surface. The magnetic coils in the opposing positions allow for shape detection of the array and identification of each electrode's position in 3D space. The magnetic sensor 123 may be a 5 degrees of freedom (DOF) sensor, wherein the degrees of freedom describe the number of axes in which a rigid body moves freely in 3D space. In some embodiments, data from the magnetic sensors is used to calculate the position of the electrode array to render an anatomical shell of the tissue. For example, in some embodiments, the shape of the deformable surface is estimated by interpolating one or more positions between the two or more magnetic sensors. Further, the position of the electrode array may be calculated from these positions. Data from the magnetic sensors may be used to generate the cardiac tissue map by sensing activity from the electrodes, thus generating a map of a region of interest in the tissue, particularly if the region is in arrhythmia.

In some embodiments, the electrode array may further comprise a plurality of pacing electrodes 125. The pacing electrodes may be positioned on the electrode array such that a refractory period may be measure. For example, in some embodiments, the pacing electrodes are positioned on a distal end of the electrode array. The pacing electrodes may also be used to stimulate activation of a cardiac rhythm. Thus, in some embodiments of the invention, the cardiac mapping catheters of the invention allow for sensing, mapping, and pacing functions on the single device.

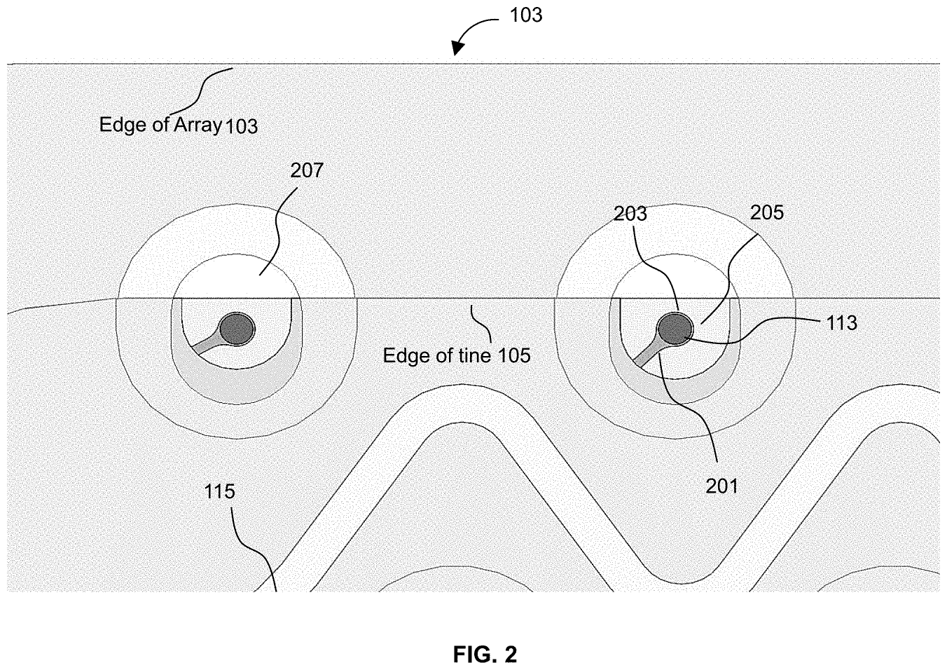

FIG. 2 illustrates individual sensing electrode detail of electrodes in an embodiment of an electrode array of the invention. As used herein, the electrode array is an ordered series or arrangement of the electrodes and separated by a known distance. The electrodes may be in the form of a plurality of stacked electrodes or an array of single electrodes.

In some embodiments, single sensing electrodes are used in the array as shown, for example, in FIG. 1. The electrodes are sufficiently thin (preferably less than about 25 μm) to enable sensing without the use of a second electrode to eliminate far-field effects. In other embodiments, the sensing electrodes are configured as an electrode pair having an orthogonal close unipolar (OCU) electrode configuration. This electrode design may consist of a two-dimensional electrode array comprising at least one stacked electrode pair (first electrode and second electrode positioned at the edge of a spline or tine. The first electrode may be referred to as the “index electrode”, and is configured to be in contact with a surface of a desired target tissue and the second electrode, which may be referred to as the “indifferent electrode” is separated from the first electrode. The electrode pair is generally arranged in an orthogonal, close, unipolar (OCU) configuration. More specifically, the common axis between the first and second electrodes (referred to as the “inter-electrode axis”) is “orthogonal” to a given surface at the target site when a recording is performed. The distance between the first and second electrodes (referred to as “inter-electrode distance”) is substantially “close”, which may be within an order of magnitude of the electrode size, such that the second electrode is “close” enough to the surface of the target tissue site to detect a bioelectrical signal. The electrode pair may be “unipolar” in that only the first electrode may be in contact with the surface of the target site.

Such a configuration addresses the limitations of existing unipolar and bipolar electrodes. In particular, recorded electrical potential of current bipolar electrodes vary with their orientation relative to the direction of a passing wavefront. Additionally, because bipolar electrodes have both electrodes on a given surface, there is potential inclusion of distinctly different electrical activity from each electrode. As such, by providing electrodes oriented perpendicular to the tissue plane, via the orthogonal close unipolar (OCU) design of the present invention, the electrode array of the present disclosure retains the superior near/far-field discrimination of common bipolar electrode recordings with the directional independence and smaller footprint of unipolar recordings. Furthermore, the unipolar electrode configuration of the present invention retains all of the spatial resolution benefits of a contact bipolar configuration, but with the additional spatial resolution enhancement conferred by a smaller footprint.

The electrode array may be in a common mode rejection (CMR) electrode configuration. A CMR electrode configuration improves the spatial resolution of electrogram recordings by decreasing the size of a region of cardiac tissue that contributes to the electrogram recording. The CMR electrode array configuration is able to accurately measure a signal derived exclusively from activation as it passes beneath a central electrode; it eliminates contribution to the local signal by activation of the tissue surrounding a central electrode. As opposed to being arranged in a stacked configuration (i.e., pairs of electrodes), the CMR array is comprised of single electrodes, which may include microelectrodes, distributed across the array at known locations such that each electrode is separated by a known distance. This embodiment allows for rapid computation of potentials across regions of a tissue. For example, electrodes in the CMR array may be useful in detecting signals of interest at bioelectrical signals propagate through tissue underneath the CMR array.

A “central” electrode in the array that detects the local activation signal works in conjunction with multiple “surrounding” contact electrodes that surround the central electrode on the array. As a local signal passes underneath the central electrode, a signal is concurrently recorded from both the central electrode and the surrounding electrodes. The signals from the surrounding electrodes are averaged, and the resulting average is subtracted from the central electrode signal. As a result, the CMR electrode array is able to accurately measure a given signal (e.g., an activation signal when used in cardiac tissue) by eliminating both far-field signal interference and near-field signal interreference from other electrodes on the array. As a result, a signal detected by the central electrode represents only the signal generated by the activation signal as it passes underneath the electrode. The CMR electrode array is thus able to leverage the benefits of unipolar electrodes and bipolar electrodes, while eliminating their drawbacks.

Accordingly, the use of simultaneously obtained electrode data according to the invention enables one to determine relative positions of measurements made by multiple electrodes in the construction of cardiac tissue mapping. Specifically, the CMR may be positioned as a distribution of electrodes on the surface of the cardiac tissue so as to allow the computation of conduction velocity. The array can be used to construct a map of cardiac rhythm and tissue properties, such as scarring.

The electrodes 113 may comprise two or more materials. For example, the electrodes may include an iridium oxide coating 203 surrounded by one or more insulating layers 205. In some embodiments, individual sensing electrodes 113 include a copper trace 201 and a copper pad that may are plated with gold. The gold thus insulates the copper components of the electrode to prevent the copper from leaching in solution. The electrode may then be further coated with iridium oxide to provide a layer of iridium oxide 203 to reduce the impedance and to achieve an adequate signal-to-noise ratio. The gold plating acts as a corrosion inhibitor for the copper trace 201. In some embodiments, the electrode comprises a slot below the insulating layer into which the copper trace is embedded such that the gold plating is on top of the copper electrode.

The electrodes may be manufactured using methods known in the art such as by using printed circuit techniques followed by coating with iridium oxide using photoresist techniques.

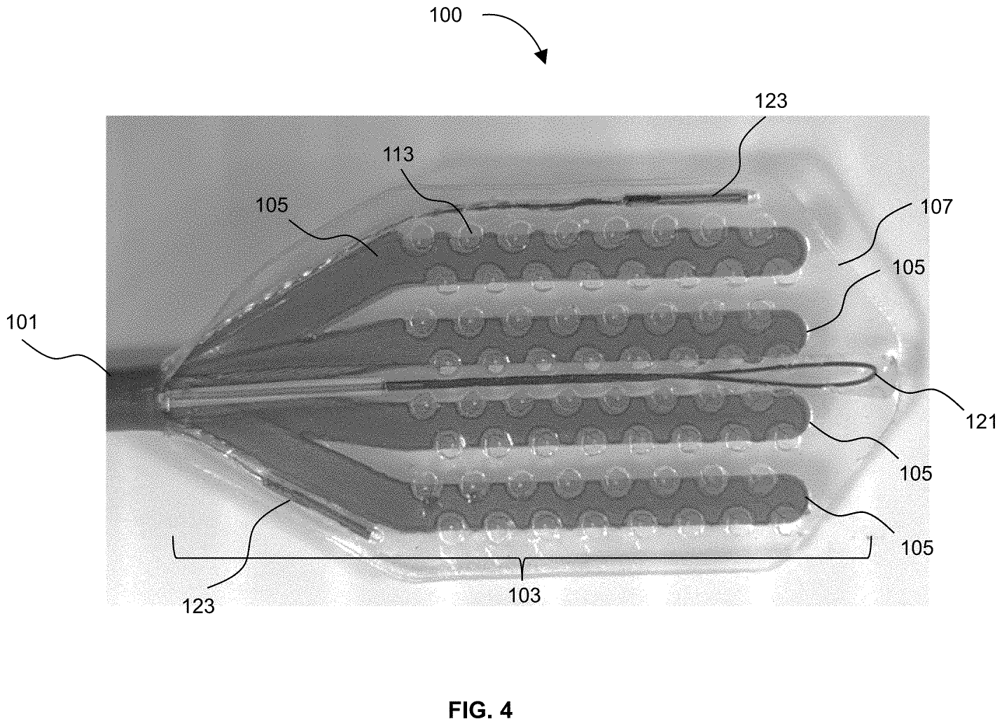

As shown in FIG. 2, in some embodiments, the electrodes of the invention are microelectrodes shaped substantially as thin discs. The electrodes, as thin discs, may be positioned as a contiguous array on the surface of the deformable surface. The electrodes may be positioned, as shown in, for example, FIG. 4, at the edge of a tine 105 or spline. Notably, the height, or alternatively the thickness, of the electrode may be less than about 150 μm. In some embodiments, the height of the electrode is less than about 25 μm. Height, as used herein, means the height of the electrode as measured from the bottom of the electrode relative to its orthogonal position on the tissue to the top of the electrode. Alternatively, the height of the electrode may also refer to the thickness of the electrode as a thin disc.

In some embodiments, the electrode array comprises a plurality of openings 207 configured to allow for fluid, such as interstitial fluid, and/or a biological matrix to pass through the electrode array. For example, the openings may be formed in the deformable surface. In some embodiments, the second layer may also include a plurality of openings to facilitate recording the potential fields generated by the electrical currents in the cardiac tissue. The openings may be, for example, holes of a diameter sufficient to allow the presence of blood. The openings allow for fluid and/or a biological matrix to enter and pass through the electrode array preventing a fluid barrier near the electrode from forming. In some embodiments, the openings comprise a diameter sufficient to allow the presence of blood.

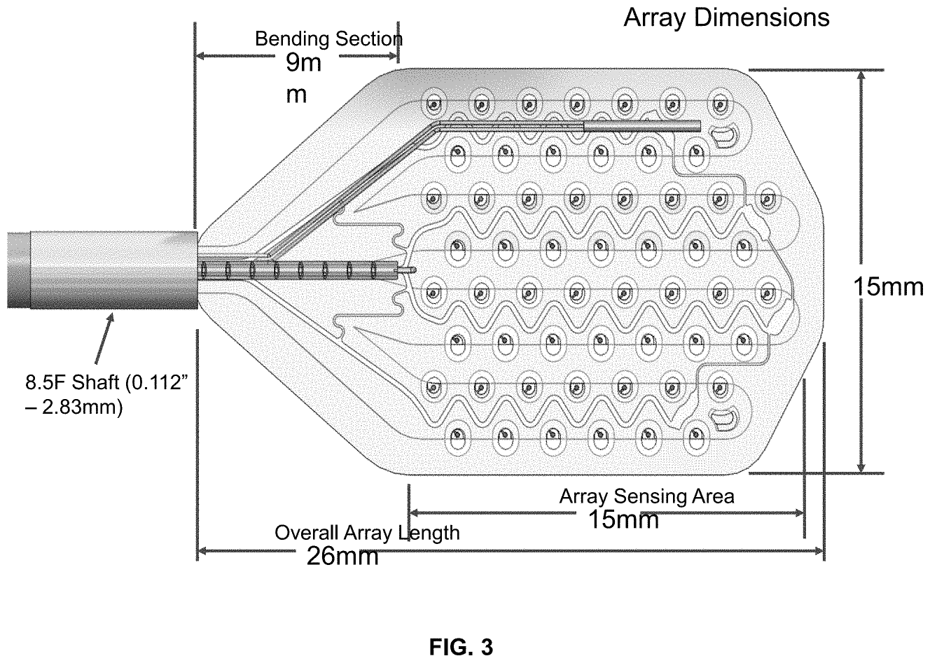

FIG. 3 illustrates non-limiting dimensions of one embodiment of a cardiac mapping catheter of the invention. The electrode array of the invention uses smaller electrodes to achieve a smaller footprint for closer spacing coupled with the flexible nature of the electrode array to improve the understanding of the dynamic behavior of the bioelectric properties of the tissue and enable a more targeted and less extensive ablation strategy. The cardiac mapping catheter of the invention may be any size, shape, or dimension sufficient to achieve the high-resolution cardiac mapping provided by devices of the invention. In non-limiting embodiments, the diameter of the electrodes may be 150 μm such that the electrode surface area is less than or equal to 0.01767 mm². The electrode array comprise 56 electrodes on each side of the deformable surface. The electrode array width may be less than or equal to 15 mm, with an overall length less than or equal to 26 mm. The array sensing area may be less than or equal to 15 mm. The cardiac mapping catheter may be used with a sheath, such as an 8.5 F steerable sheath with a diameter ranging from about 0.122 inches to about 2.83 mm.

FIG. 4 illustrates one embodiment of a cardiac mapping catheter of the present invention.

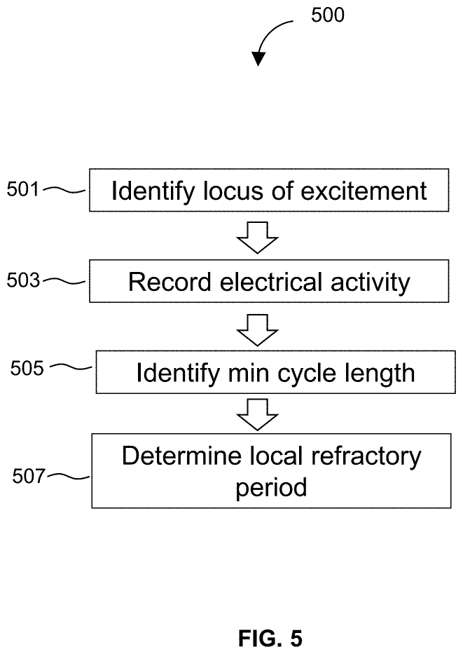

FIG. 5 diagrams an embodiment of a method of mapping cardiac tissue properties 500. The cardiac mapping catheters described herein may be used for improved mapping of cardiac tissue properties. The method includes providing a device of the invention, for example, a cardiac mapping catheter comprising an electrode array comprising a flexible support frame and a deformable surface, wherein upon deployment from a sheath the electrode array conforms to a shape of a tissue to lie substantially flat when deployed against the tissue. Further, the method includes identifying 501 a locus of excitation in cardiac tissue, recording 503 the electrical activity in that location, identifying the minimum cycle length during fibrillation 505 to determine the local refractory period 507. Thus, a 3D map of a distribution of minimum cycle length is created. The locus of excitation may be an area that is electrically active.

Alternatively, one can stimulate the heart from one or more of the electrodes on the catheter and record local capture to determine the shortest stimulus coupling interval capable of local capture to determine local refractory period. This method allows detection of refractory period even in the presence of an “excitable gap” (a period when a cell has recovered the capacity to be excited but has not been stimulated by an incoming excitation wave) during fibrillation.

In some embodiments, the identifying step comprises identifying via determination of the minimum pacing interval resulting in local excitation. As described above, in some embodiments, electrode array comprises a plurality of single electrodes arranged in an orthogonal, close, unipolar configuration. Alternatively, in some embodiments, the electrode array comprises a plurality of electrode pairs arranged in an orthogonal, close, unipolar configuration as previously described.

### INCORPORATION BY REFERENCE

References and citations to other documents, such as patents, patent applications, patent publications, journals, books, papers, web contents, have been made throughout this disclosure.

All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A cardiac mapping catheter comprising:
an electrode array comprising a flexible support frame and a deformable surface, wherein upon deployment from a sheath the electrode array conforms to a shape of a tissue to lie substantially flat when deployed against the tissue.

2. The cardiac mapping catheter of claim 1, wherein the deformable surface is a flexible membrane.

3. The cardiac mapping catheter of claim 2, wherein the flexible membrane comprises a first layer having a first durometer number, and a second layer having a second durometer number lower than said first durometer number.

4. The cardiac mapping catheter of claim 1, wherein the electrode array comprises an array of single electrodes.

5. The cardiac mapping catheter of claim 1, wherein the flexible support frame comprises a shape memory metal.

6. The cardiac mapping catheter of claim 1, wherein the electrode array comprises an array of electrode pairs.

7. The cardiac mapping catheter of claim 6, wherein the electrode pairs are arranged in an orthogonal close unipolar configuration.

8. The cardiac mapping catheter of claim 7, further comprising a plurality of tines wherein the electrodes of the electrode array are positioned on each of a first side and a second side of the tines.

9. The cardiac mapping catheter of claim 4, wherein the single electrodes have a thickness of about 25 μm.

10. The cardiac mapping catheter of claim 4, wherein the single electrodes are substantially shaped as a thin disc with a height of less than or equal to about 0.25 μm.

11. The cardiac mapping catheter of claim 4, wherein the single electrodes comprise a gold-plated copper trace coated with iridium oxide and surrounded by an insulating layer.

12. The cardiac mapping catheter of claim 4, wherein the electrode array comprises a plurality of openings configured to allow fluid to pass through the electrode array.

13. The cardiac mapping catheter of claim 1, wherein the deformable surface is furled when retracted back into the sheath.

14. The cardiac mapping catheter of claim 13, wherein the deformable surface is substantially s-shaped when furled within the sheath.

15. The cardiac mapping catheter of claim 1, wherein said catheter further comprises two or more magnetic tracking sensors on opposing sides of the deformable surface.

16. The cardiac mapping catheter of claim 1, wherein the electrode array further comprises a plurality of pacing electrodes.
